(19) United States
(12) Patent Application Publication
(10) Pub. No.: US 2004/0087771 A1
(43) Pub. Date: May 6, 2004

(54) Antimicrobial peptides of the family of defensins, polynucleotides encoding said peptides, transformed vectors and organisms containing them

(76) Inventors: Mireille Lamberty, Strasbourg (FR); Philippe Bulet, Vendenheim (FR); Marie-Pascale Latorse, Sourcieux Les Mines (FR); Jules Hoffmann, Strasbourg (FR)

Correspondence Address: Connolly Bove Lodge & Hutz, P O Box 2207, Wilmington, DE, 19899-2207, US

(21) Appl. No.: 10/311,948

(22) PCT Filed: Jun. 27, 2001
(86) PCT No.: PCT/FR01/02028

(30) Foreign Application Priority Data
Jun. 29, 2000 (FR) 00/08374

(51) Int. Cl. C07K 14/195; C12N 1/21; C07H 21/04

(52) U.S. Cl. 530/350; 536/23.7; 435/69.1; 435/252.3; 435/320.1

(57) ABSTRACT

The invention concerns novel antimicrobial peptides of the family of defensins, in particular antifungal, called termicines, polynucleotides encoding said peptides, vectors containing them for transforming a host organism and the method for transforming said organism. The invention also concerns transformed organisms, in particular yeast producing termicine, or plant cells and plants, the termicines produced by the transformed plats providing them with resistence to fungus-mediated diseases. The invention further concerns the use of termicines as medicine and pharmaceutical compositions containing them.

The present invention relates to novel antimicrobial peptides of the family of defensins, in particular antifungal peptides, called termicins, to polynucleotides encoding these peptides, to vectors containing them for the transformation of a host organism, and to the method for transforming said organism.

The invention also relates to transformed organisms, in particular yeasts producing termicin, or plant cells and plants, the termicins produced by the transformed plants conferring on them resistance to diseases, in particular of fungal origin.

The invention-also relates to the use of the termicins as a medicinal product, and to the pharmaceutical compositions comprising them.

In the field of plant health, as in that of animal health, there is a permanent need to produce novel molecules for combating infections of fungal origin. In plant health, the protection of crops against fungal diseases essentially involves spraying synthetic fungicides onto said crops. However, there is today a growing need to make plants resistant to diseases, in particular fungal diseases, in order to decrease, or even to avoid, resorting to treatments with antifungal protection products, with a view to protecting the environment. A means of increasing this resistance to diseases consists in transforming the plants in such a way that they produce substances able to defend them against these diseases.

In the field of human and animal health, opportunistic fungal infections exist for which no really effective treatment is available at the current time. In particular, this is the case of life-threatening invasive mycoses which affect hospitalized patients whose immune system is suppressed following a transplant, chemotherapy or HIV infection. By comparison with the arsenal of antibacterial agents, the current panoply of antifungal agents is very limited. There is therefore a real need to characterize and develop novel classes of antifungal substances.

The technical problem of the present invention therefore consists in isolating novel antifungal peptides, which peptides will find applications in the fields of crop protection, and also in those of human and animal health and nutrition.

In invertebrates, in particular in insects, a certain number of substances are known which are naturally produced by these organisms and which confer on them protection against pathogenic agents of bacterial or fungal origin. These substances are generally peptides, which are termed antibacterial, antifungal or antimicrobial depending on whether they have preferential activity with respect to bacteria or fungi, or mixed activity with respect to these two types of pathogen. For the purpose of the present invention, the terms “bactericidal” or “fungicidal” are intended to mean both bactericidal and fungicidal properties per se, and bacteriostatic or fungistatic properties.

Among the known insect peptides, mention may be made of those described in patent applications WO 97/30082, WO 99/24594, WO 99/02717, WO 99/53053, and WO 99/91089. The difficulty in finding novel peptides in insects comes from the fact that various classes of peptide exist which are not found in all insects, and that peptides belonging to the same class may have different properties depending on the insects from which they are isolated (Dimarcq et al., 1998, Biopolymers [Peptide science], Vol. 47, 465-477; Bulet et al., 1999, Dev. Comp. Immunol., Vol. 23, 329-344).

With regard to the difficulties mentioned above, it emerges that the current state of the art does not make it possible to deduce a biological activity only from the peptide structure of the known antimicrobial peptides. Consequently, it is therefore relatively ineffective to use the peptide sequence of known peptides as a basis for seeking to create a novel peptide with a desired antimicrobial activity. It is also relatively ineffective to use the nucleotide sequence encoding an antimicrobial peptide described in the state of the art, as a probe to search for similar sequences in cDNA libraries derived from other organisms, except when these other organisms are part of a restricted zoological group, for example a family, within which there exists only a small molecular variability. The solution to the technical problem of the present invention therefore lies in the isolation of peptides identified, initially, preferentially by their antimicrobial properties rather than by their structures. This solution is obtained with the definition of in vitro assays for bacterial, fungal or yeast growth inhibition, which assays make it possible to screen extracts of a large number of organisms, in particular insects, for the presence of activities with respect to at least one of these assays.

In defining a strategy for isolating novel peptides, it therefore appeared to be important to direct the research toward insects living in hostile environments in contact with potential pathogenic microorganisms, in order to increase the chances of finding insects in which the arsenal of peptides is particularly well developed since it is adapted to these environments. In addition, in order to target a particular activity, it is also possible to direct the screening toward insects which are highly suspected, because of their habits, of possessing peptides having preferential activity with respect to a type of microorganism. It is the latter strategy which has been implemented in the present invention in order to answer the technical problem stated above. Specifically, in order to isolate novel antifungal peptides, it was chosen to screen insects living in close contact with fungi.

The solution to the technical problem consisting in isolating novel antifungal peptides has therefore been obtained by isolating novel peptides, termicins, isolated from insects living in symbiotic contact with a fungus, in particular from fungus-growing termites of the family Macrotermitinae. This termite family lives in close contact with a symbiotic basidiomycete fungus of the Termitomyces genus, which provides it with efficient digestion of lignocellulose materials. In particular, a characteristic termicin is isolated from the termite Pseudacanthotermes spiniger. In general, a termicin is a peptide belonging to the insect defensin class. The insect defensin class includes mainly antibacterial and/or antifungal peptides characterized in that they contain six cysteines connected to one another by three disulfide bridges.

A subject of the invention is therefore novel peptides, termicins. As anticipated by the strategy described above, termicins exhibit mainly fungicidal activity, in particular against the filamentous fungi responsible for plant diseases and the fungi involved in human and animal pathology. After analysis, it appears that termicins also possess activity with respect to the yeasts involved in human pathology, and also lytic or static properties on Gram-positive bacteria. After having synthesized a gene encoding a termicin, it was found that this gene could be inserted into a host organism, such as a yeast or a plant, in order to express said termicin in said host organism, and either to produce purified or unpurified termicin or to confer on said host organism properties of resistance to fungal diseases, providing a particularly advantageous solution to the problems stated above.

The present invention also relates to polynucleotides encoding a termicin as defined above. According to the present invention, the term “polynucleotide” is intended to mean a natural or artificial nucleotide sequence which may be of the DNA or RNA type, preferably of the DNA type, in particular double-stranded.

According to particular embodiments of the invention, the polynucleotides can either be synthesized artificially, or can correspond to the polynucleotides of the insect from which they are isolated, or else correspond to fragments derived from these polynucleotides, adapted for expression of the termicin in the host organism where said termicin will be expressed. The polynucleotides can be obtained according to standard methods of isolation and purification, or else by synthesis according to the usual techniques of successive hybridizations of synthetic oligonucleotides. These techniques are in particular described by Ausubel et al. (1987, Current Protocols in Molecular Biology, eds. Greene, Publ. Wiley & Sons).

According to a particular embodiment of the invention, the polynucleotides encoding the termicin comprise polynucleotides encoding the peptide sequence described by the sequence identifier SEQ ID No. 2. It is well known to those skilled in the art that this definition includes all the polypeptides which, although comprising nucleotide sequences which are different as a result of the degeneracy of the genetic code, encode the same amino acid sequence, which is represented by the sequence identifier SEQ ID No. 2.

The present invention also comprises isolated polynucleotides encoding termicins and capable of hybridizing selectively to one of the polynucleotides described above. According to the invention, the expression “polynucleotide capable of hybridizing selectively” is intended to mean the polynucleotides which, using one of the usual methods of the state of the art (Sambrook et al., 1989, Molecular Cloning: A Laboratory Manual, Nolan C. ed., New York: Cold Spring Harbor Laboratory Press), hybridize with the polypeptides above at a level significantly greater than the background noise. The background noise may be associated with the hybridization of other polynucleotides present, in particular of other cDNAs present in a cDNA library. The level of the signal generated by the interaction between the polynucleotide capable of hybridizing selectively and the polynucleotides defined by the sequence ID above according to the invention is generally 10 times, preferably 100 times, stronger than that of the interaction of the other DNA sequences generating the background noise. The level of interaction can be measured for example by labeling the probe with radioactive elements, such as ³²P. Selective hybridization is generally obtained using very severe medium conditions (for example 0.03M NaCl and 0.03M sodium citrate at approximately 50° C.-60° C.).

The invention also comprises isolated polynucleotides encoding termicins and homologous to the polynucleotides described above. According to the invention, the term “homologous” is intended to mean polynucleotides having one or more sequence modifications with respect to the nucleotide sequences described above, and encoding a termicin the properties of which are not significantly modified. These modifications can be obtained according to the usual techniques of mutation leading in particular to the addition, deletion or substitution of one or more nucleotides with respect to the sequences of the invention. Advantageously, the degree of homology will be at least 70% relative to the sequences of the invention, preferably at least 80%, more preferentially at least 90%. The methods for measuring and identifying homologies between nucleic acid sequences are well known to those skilled in the art. Use may be made, for example, of the PILEUP or BLAST programs (in particular Altschul et al., 1993, J. Mol. Evol. 36: 290-300; Altschul et al., 1990, J. Mol. Biol. 215: 403-10).

The present invention also relates to fragments of polynucleotides described above. The term “fragment” denotes in particular a fragment of at least 20 nucleotides, in particular of at least 50 nucleotides, and preferably of at least 100 nucleotides.

According to a particular embodiment of the invention, the polynucleotide according to the invention is represented by the sequence identifier SEQ ID No. 1.

The present invention also relates to polynucleotides comprising at least one of the polynucleotides as described above.

All the polynucleotides described above encode termicins. Consequently, the invention therefore extends to all the peptides encoded by all these polynucleotides.

According to a particular embodiment of the invention, a termicin is a peptide comprising the peptide sequence described by the sequence identifier SEQ ID No. 2 or a fragment of this sequence. The term “fragment” is essentially intended to mean a biologically active fragment, i.e. a fragment of the sequence of a termicin, which has the same antimicrobial activity as a complete termicin.

The terminal NH² residue of a termicin may exhibit a posttranslational modification, for example an acetylation, in the same way that the C-terminal residue may exhibit a posttranslational modification, for example an amidation.

In addition, a termicin as described in the present invention differs from the insect defensins of the state of the art by its peptide structure, and in particular differs from another insect defensin, heliomicin described in patent application WO 99/53053, by the structural characteristic of having more than 9 amino acid residues between cysteines No. 3 and 4.

According to a preferential embodiment of the invention, the cysteine residues of a termicin form at least one intramolecular disulfide bridge, preferably three disulfide bridges. According to a preferential embodiment of the invention, the disulfide bridges are established between cysteine residues 1 and 4, 2 and 5 and 3 and 6.

The present invention also relates to a chimeric gene comprising, functionally linked to one another, at least one promoter which is functional in a host organism, a polynucleotide encoding a termicin as defined in the present invention, and a terminator element which is functional in this same host organism. The various elements that a chimeric gene may contain are, firstly, elements regulating transcription, translation and maturation of proteins, such as a promoter, a sequence encoding a signal peptide or a transit peptide, or a terminator element constituting a polyadenylation signal and, secondly, a polynucleotide encoding a protein. The expression “functionally linked to one another” means that said elements of the chimeric gene are linked to one another in such a way that the functioning of one of these elements is affected by that of another. By way of example, a promoter is functionally linked to a coding sequence when it is capable of affecting the expression of said coding sequence. The construction of the chimeric gene according to the invention and the assembly of its various elements can be carried out using techniques well known to those skilled in the art, in particular those described in Sambrook et al. (1989, Molecular Cloning: A Laboratory Manual, Nolan C. ed., New York: Cold Spring Harbor Laboratory Press). The choice of the regulatory elements constituting the chimeric gene is essentially made as a function of the host species in which they must function, and those skilled in the art are capable of selecting regulatory elements which are functional in a given host organism. The term “functional” is intended to mean capable of functioning in a given host organism.

The promoters that the chimeric gene according to the invention may contain are either constitutive or inducible. It also appears to be important for the chimeric gene to additionally comprise a signal peptide or a transit peptide which makes it possible to control and orient the termicin production specifically in a part of the host organism, such as, for example, the cytoplasm, a particular compartment of the cytoplasm, or the cell membrane or, in the case of plants, in a particular type of cellular compartment or of tissue or in the extracellular matrix.

According to one embodiment, the transit peptide may be a chloroplast or mitochondrial addressing signal, which is then cleaved in the chloroplasts or the mitochondria.

According to another embodiment of the invention, the signal peptide may be an N-terminal signal or “prepeptide”, optionally in combination with a signal responsible for retention of the protein in the endoplasmic reticulum, or a vacuolar addressing peptide or “propeptide”. The endoplasmic reticulum is the cellular compartment where operations for maturation of the protein produced are carried out, such as, for example, cleavage of the signal peptide.

The transit peptides can be either single or double. The double transit peptides are optionally separated by an intermediate sequence, i.e. they comprise, in the direction of transcription, a sequence encoding a transit peptide of a plant gene encoding an enzyme located in plastids, a portion of sequence of the mature N-terminal portion of a plant gene encoding an enzyme located in plastids, and then a sequence encoding a second transit peptide of a plant gene encoding an enzyme located in plastids. Such double transit peptides are, for example, described in patent application EP 0 508 909.

Signal peptides of use according to the invention which may be mentioned include in particular the signal peptide of the tobacco PR-1α gene described by Cornelissen et al., (1987, Nucleic Acid Res. 15. 6799-6811), in particular when the chimeric gene according to the invention is introduced into plant cells or plants, or the signal peptide of the Mat α1 factor precursor (Brake et al., 1985, In: Gething M. -J. (eds.); Protein transport and secretion, pp. 103-108, Cold Spring Harbor Laboratory Press, New York), where the chimeric gene according to the invention is introduced into yeasts.

The present invention also relates to a vector containing a chimeric gene according to the invention. The vector according to the invention is of use for transforming a host organism and expressing a termicin in this host organism. This vector may be a plasmid, a cosmid, a bacteriophage or a virus. In general, the main qualities of this vector should be an ability to persist and to self-replicate in the host organism's cells, in particular by virtue of the presence of an origin of replication, and to express a termicin therein. The choice of such a vector and also the techniques for inserting therein the chimeric gene according to the invention are widely described in Sambrook et al. (1989, Molecular Cloning: A Laboratory Manual, Nolan C. ed., New York: Cold Spring Harbor Laboratory Press) and are part of the general knowledge of those skilled in the art. Advantageously, the vector used in the present invention also contains, in addition to the chimeric gene of the invention, a chimeric gene containing a selectable marker. This selectable marker makes it possible to select the host organisms effectively transformed, i.e. those which have incorporated the vector. According to a particular embodiment of the invention, the host organism to be transformed is a plant. According to another embodiment, the host organism is a microorganism, in particular a yeast. Among the selectable markers which can be used, mention may be made of markers containing genes for resistance to antibiotics, such as, for example, that of the hygromycin phosphotransferase gene (Gritz et al., 1983, Gene 25: 179-188), but also markers containing genes for tolerance to herbicides, such as the bar gene (White et al., NAR 18: 1062, 1990) for tolerance to bialaphos, the EPSPS gene (U.S. Pat. No. 5,188,642) for tolerance to glyphosate or the HPPD gene (WO 96/38567) for tolerance to isoxazoles. Mention may also be made of genes encoding readily identifiable enzymes such as the GUS enzyme, or genes encoding pigments or enzymes which regulate pigment production in the transformed cells. Such selectable marker genes are in particular described in patent applications WO 91/02071, WO 95/06128, WO 96/38567, and WO 97/04103.

The present invention also relates to transformed host organisms containing a vector as described above. The term “host organism” is intended to mean any lower or higher monocellular or pluricellular organism into which the chimeric gene according to the invention can be introduced, to produce termicin. They are in particular bacteria, for example E. coli, yeasts, in particular of the Saccharomyces, Kluyveromyces, or Pichia genus, fungi, in particular Aspergillus, a baculovirus, or preferably plant cells and plants.

According to the invention, the term “plant cell” is intended to mean any cell derived from a plant and able to constitute undifferentiated tissues such as calluses, differentiated tissues such as embryos, parts of plants, plants or seeds.

According to the invention, the term “plant” is intended to mean any differentiated multicellular organism capable of photosynthesis, in particular monocotyledons or dicotyledons, more particularly crop plants possibly intended for animal or human nutrition, such as maize, wheat, rapeseed, soybean, rice, sugar cane, beetroot, tobacco or cotton.

The term “transformed host organism” is intended to mean a host organism which has incorporated into its genome the chimeric gene of the invention and consequently produces a termicin in its tissues or in a culture medium. Those skilled in the art can use one of the many known methods of transformation to obtain the host organisms according to the invention.

One of these methods consists in bringing the cells to be transformed into contact with polyethylene glycol (PEG) and the vectors of the invention (Chang and Cohen, 1979, Mol. Gen. Genet. 168(1), 111-115; Mercenier and Chassy, 1988, Biochimie 70(4), 503-517). Electroporation is another method, which consists in subjecting the cells or tissues to be transformed and the vectors of the invention to an electric field (Andreason and Evans, 1988, Biotechniques 6(7), 650-660; Shigekawa and Dower, 1989, Aust. J. Biotechnol. 3(1), 56-62). Another method consists in directly injecting the vectors into the host cells or tissues by microinjection (Gordon and Ruddle, 1985, Gene 33(2), 121-136). Advantageously, the “biolistic” method may be used. It consists in bombarding cells or tissues with particles onto which the vectors of the invention are adsorbed (Bruce et al., 1989, Proc. Natl. Acad. Sci. USA 86(24), 9692-9696; Klein et al., 1992, Biotechnology 10(3), 286-291; U.S. Pat. No. 4,945,050). Preferentially, plant transformation will be carried out using bacteria of the Agrobacterium genus, preferably by infecting the cells or tissues of said plants with A. tumefaciens (Knopf, 1979, Subcell. Biochem. 6, 143-173; Shaw et al., 1983, Gene 23(3): 315-330) or A. rhizogenes (Bevan and Chilton, 1982, Annu. Rev. Genet. 16: 357-384; Tepfer and CasseDelbart, 1987, Microbiol. Sci. 4(1), 24-28). Preferentially, the transformation of plant cells with Agrobacterium tumefaciens is carried out according to the protocol described by Ishida et al. (1996, Nat. Biotechnol. 14(6), 745-750).

Those skilled in the art will choose the appropriate method as a function of the nature of the host organism to be transformed.

According to a particular embodiment of the invention, the present invention therefore also relates to transformed microorganisms containing a chimeric gene according to the invention and expressing the termicin. The transformation of microorganisms makes it possible to produce termicin on a semi-industrial or industrial scale. The microorganism to be transformed may be a yeast, a fungus, a bacterium or a virus. Depending on the microorganism to be transformed, those skilled in the art will be able to select the regulatory elements for the chimeric gene which make it possible to optimize the termicin production. These regulatory elements are in particular promoter sequences, transcription activators, signal or transit peptides, terminator sequences and start and stop codons.

Preferentially, the transformed host organism is a yeast. By way of example, the transformation of a yeast can be carried out with an expression vector comprising a polynucleotide encoding the termicin and the following elements:

markers for selecting the transformants. Preferably, the ura-3 gene is used for yeast and the gene which confers resistance to ampicillin is used for E. coli;

a nucleic acid sequence for replication (origin of replication) of the plasmid in the yeast. Preferably, the origin of replication of the yeast 2 μ plasmid is used;

a nucleic acid sequence for replication (origin of replication) of the plasmid in E. coli;

a chimeric gene according to the invention consisting

(1) of a promoter regulatory sequence. Any promoter sequence for a gene which is naturally expressed in yeast may be used. Preferably, use is made of the promoter of the S. cerevisiae Mfα1 gene as described in Betz et al. (1987, J. Biol. Chem., 262, 546-548) or Reichhart et al. (1992, Invert. Reprod. Dev., 21, 15-24),

(2) of a sequence encoding a signal peptide (or prepeptide) in combination with an addressing peptide (or propeptide). These regions are important for correct secretion of the peptide. Preferably, use is made of the sequence encoding the prepropeptide of the MFα1 factor precursor as described in Betz et al. (1987, J. Biol. Chem., 262, 546-548) or Reichhart et al. (1992, Invert. Reprod. Dev., 21, 15-24),

(3) of a polynucleotide according to the invention,

(4) of a terminator regulatory sequence or a polyadenylation sequence. Preferably, the S. cerevisiae phosphoglycerate kinase (PGK) terminator is used. In the expression cassette, the sequence encoding the termicin is inserted downstream of the prepro sequence and upstream of the PGK terminator.

These elements have been described in several publications, including Reichhart et al. (1992, Invert. Reprod. Dev., 21, pp. 15-24), Michaut et al. (1996, FEBS Letters, 395, pp. 6-10) and Lamberty et al. (1999, JBC, 274, pp. 9320-9326).

Preferentially, yeasts of the S. cerevisiae species are transformed with the expression vector using the lithium acetate method (Ito et al., 1993, J. Bacteriol, 153. pp. 163-168). The transformed yeasts are selected on a selective agar medium which does not contain any uracil. The mass production of the transformed yeasts is carried out by culture for 24 h to 48 h in a selective liquid medium.

The present invention therefore also relates to a method for preparing the termicin, comprising the steps of culturing a transformed microorganism comprising a gene encoding the termicin as defined above, in a suitable culture medium, and then extracting and purifying, totally or partially, the termicin obtained. Preferentially, the termicin-producing microorganism used is a transformed yeast comprising a chimeric gene according to the invention.

Preferably during the extraction of the termicin produced by the yeasts, the yeasts are removed by centrifugation and the culture supernatant is brought into contact with an acidic solution, which may be a solution of an inorganic or organic acid, such as, for example, hydrochloric acid or acetic acid. The extract obtained is then centrifuged under cold conditions, at a rate of 4 000 to 10 000 rpm at 4° C., for 30 to 60 min.

Purification of the termicin may be preceded by a step of fractionation of the supernatant obtained subsequent to the extraction. Preferably, during the fractionation step, the extract is deposited on reverse phase in order to perform a solid-phase extraction. The water-soluble molecules are washed with a dilute acid solution and the hydrophobic molecules are eluted with a suitable eluent. Preferentially, trifluoroacetic acid is used for the washing and an eluent containing increasing amounts of acetonitrile in dilute acid solution is used.

Advantageously, a second solid-phase extraction step is carried out, and preferentially on ion exchange phase. The molecules not retained are washed in a saline buffer at acid pH and the cationic molecules are eluted with a solution containing an increasing concentration of salts. The quality required for correct attachment of the molecules is obtained with an ammonium acetate buffer at a concentration of less than 100 μM. Preferentially, an eluent containing a saline chaotropic agent in buffered solution is used.

Preferably, the purification of the termicin is carried out in a reverse-phase HPLC step with a suitable eluent which may be different from or identical to that of the preceding reverse phase. The various steps of the purification are followed by an assay for fungal and bacterial growth inhibition in liquid medium. Preferably, the assays are carried out with the fungus Neurospora crassa, and the bacterium Micrococcus luteus.

The sequence of the termicin produced by the transformed yeasts is analyzed according to the Edman degradation sequencing method and by mass spectrometry.

The structural characterization is performed directly on the peptide produced, on the peptide modified by reduction/alkylation, and also on fragments of the peptide. The peptide sequence and the molecular mass of the termicin produced, and also the minimum inhibitory concentration (MIC) with respect to the filamentous fungus Neurospora crassa were compared with those of a reference termicin, the native termicin extracted from whole bodies of Pseudacanthotermes spiniger. The results show that the two molecules have the same primary structure, with the exception of the presence of a supplementary C-terminal amino acid in the recombinant molecule, namely a peptide residue consisting of an amino acid, preferably glycine (Gly) to replace the C-terminal amidation of the peptide residue represented by the amidated C-terminal amino acid of the natural molecule, namely the peptide residue consisting of the amino acid—Arg-amide. Determination of the position of the disulfide bridges indicates that the arrangement of the disulfide bridges is identical in the two peptides, the native peptide and the peptide produced by the transformed microorganism.

The invention also relates to transformed plants containing a chimeric gene according to the invention and expressing a termicin according to the invention in their tissues, said termicin conferring on these plants resistance to pathogenic organisms. The chimeric gene used to obtain transformed plants according to the invention may contain a constitutive promoter or an inducible promoter. Promoters which may be used include any promoter of a gene which is expressed naturally in plants, in particular a promoter of bacterial, viral or plant origin. Among the constitutive promoters which may be used in the chimeric gene of the present invention, mention may be made, by way of example, of bacterial promoters, such as that of the octopine synthase gene or that of the nopaline synthase gene, of viral promoters, such as that of the gene controlling transcription of the 19S or 35S RNAs of the cauliflower mosaic virus (Odell et al., 1985, Nature, 313, 810-812), or the promoters of the cassava vein mosaic virus (as described in patent application WO 97/48819). Among the promoters of plant origin, mention will be made of the promoter of the ribulose-biscarboxylase/oxygenase (RuBisCO) small subunit gene, the promoter of a histone gene as described in application EP 0 507 698, or the promoter of a rice actin gene (U.S. Pat. No. 5,641,876). Mention may also be made of the regulatory element defined by the functional association of a histone gene promoter associated with an actin gene intron as described in patent application WO 99/34005.

According to another particular embodiment of the invention, the chimeric gene contains an inducible promoter. An inducible promoter is a promoter which only functions, i.e. which only induces expression of a coding sequence, when it is itself induced by an inducing agent. This inducing agent is generally a substance which can be synthesized in the host organism subsequent to a stimulus external to said organism, this external stimulus possibly being a pathogenic agent for example. The inducing agent may also be a substance external to this host organism, capable of penetrating into this host organism. Advantageously, the promoter used in the present invention is inducible subsequent to an attack on the host organism by a pathogenic agent. Such promoters are known, such as, for example, the promoter of the plant O-methyl transferase class II (COMT II) gene described in patent application FR 99 03700, the Arabidopsis PR-1 promoter (Lebel et al., 1998, Plant J. 16(2):223-233), the EAS4 promoter of the tobacco sesquiterpene synthase gene (Yin et al., 1997, Plant Physiol. 115(2), 437-451), or the promoter of the gene encoding 3-hydroxy-3-methylglutaryl coenzyme A reductase (Nelson et al., 1994, Plant Mol. Biol. 25(3): 401-412).

According to the invention, the chimeric gene may also contain, in combination with the promoter regulatory sequence, other regulatory sequences, which are located between the promoter and the coding sequence, such as transcription activators (enhancers), for instance the translation activator of the tobacco mosaic virus (TMV) described in application WO 87/07644, or the tobacco etch virus (TEV) described by Carrington and Freed (1990, J. Virol. 64, 1590-1597), for example. It may also contain a signal peptide or a transit peptide as described above.

Among the terminator elements which may be used in the chimeric gene of the present invention, mention may be made, by way of example, of the nos terminator element of the gene encoding Agrobacterium tumefaciens nopaline synthase (Bevan et al., 1983, Nucleic Acids Res. 11(2), 369-385), or the terminator element of a histone gene as described in application EP 0 633 317.

For the methods for transforming the plant cells and for regenerating the plants, mention will in particular be made of the following patents and patent applications: U.S. Pat. No. 4,459,355, U.S. Pat. No. 4,536,475, U.S. Pat. No. 5,464,763, U.S. Pat. No. 5,177,010, U.S. Pat. No. 5,187,073, EP 267,159, EP 604 662, EP 672 752, U.S. Pat. No. 4,945,050, U.S. Pat. No. 5,036,006, U.S. Pat. No. 5,100,792, U.S. Pat. No. 5,371,014, U.S. Pat. No. 5,478,744, U.S. Pat. No. 5,179,022, U.S. Pat. No. 5,565,346, U.S. Pat. No. 5,484,956, U.S. Pat. No. 5,508,468, U.S. Pat. No. 5,538,877, U.S. Pat. No. 5,554,798, U.S. Pat. No. 5,489,520, U.S. Pat. No. 5,510,318, U.S. Pat. No. 5,204,253, U.S. Pat. No. 5,405,765, EP 442 174, EP 486 233, EP 486 234, EP 539 563, EP 674 725, WO 91/02071 and WO 95/06128.

According to the present invention, the chimeric gene may also be associated with a selectable marker suitable for the transformed host organism. Such selectable markers are well known to those skilled in the art. It may be a gene for resistance to antibiotics, or else a gene for tolerance to herbicides for plants. Such genes for tolerance to herbicides are well known to those skilled in the art, and are in particular described in patent applications EP 115 673, WO 87/04181, EP 337 899, WO 96/38567 and WO 97/04103.

The transformed plants according to the invention also include the transformed plants derived from growing and/or crossing the regenerated plants above, and also the seeds of transformed plants.

The plants thus transformed are resistant to certain diseases, in particular to certain fungal or bacterial diseases, preferably to fungal diseases such as those caused, for example, by a fungus of the Cercospora genus, in particular Cercospora fijensis, of the Septoria genus, in particular Septoria nodorum or Septoria tritici, of the Fusarium genus, in particular Fusarium nivale or Fusarium graminearum, of the Botrytis genus, in particular Botrytis cinerea, or of the Rhizoctonia genus, in particular Rhizoctonia solani.

Of course, the transformed cells and plants according to the invention may comprise, in addition to a chimeric gene according to the invention, at least one other chimeric gene containing a polynucleotide encoding a protein of interest. Among the polynucleotides encoding a protein of interest, mention may be made of polynucleotides encoding an enzyme for resistance to a herbicide, for example the polynucleotide encoding the bar enzyme (White et al., NAR 18:1062, 1990) for tolerance to bialaphos, the polynucleotide encoding the EPSPS enzyme (U.S. Pat. No. 5,188,642; WO 97/04103) for tolerance to glyphosate or else the polynucleotide encoding the HPPD enzyme (WO 96/38567) for tolerance to isoxazoles. Mention may also be made of a polynucleotide encoding an insecticidal toxin, for example a polynucleotide encoding a toxin of the bacterium Bacillus thuringiensis (for example, see International Patent Application WO 98/40490). Other polynucleotides for resistance to diseases may also be contained in these plants, for example a polynucleotide encoding the oxalate oxidase enzyme as described in patent application EP 0 531 498 or U.S. Pat. No. 5,866,778, or a polynucleotide encoding another antibacterial and/or antifungal peptide, such as those described in patent applications WO 97/30082, WO 99/24594, WO 99/02717, WO 99/53053 and WO 99/91089. Mention may also be made of polynucleotides encoding agronomic characteristics of the plant, in particular a polynucleotide encoding a delta-6 desaturase enzyme as described in patents U.S. Pat. No. 5,552,306 and U.S. Pat. No. 5,614,313, and patent applications WO 98/46763 and WO 98/46764, or a polynucleotide encoding a serine acetyl transferase (SAT) enzyme as described in patent applications WO 00/01833 and PCT/FR 99/03179.

The other sequences may be integrated by means of the same vector comprising a chimeric gene, which comprises a first sequence encoding the termicin and at least one other sequence encoding another peptide or protein of interest.

They may also be integrated by means of another vector comprising at least said other sequence, according to the usual techniques defined above.

The plants according to the invention may also be obtained by crossing parents, one carrying the gene according to the invention encoding the termicin, the other carrying a gene encoding at least one other peptide or protein of interest.

The present invention also relates to a method for growing the transformed plants according to the invention, the method consisting in planting the seeds of said transformed plants in an area of a field suitable for growing said plants, in applying to said area of said field an agrochemical composition, without substantially affecting said seeds or said transformed plants, then harvesting the plants grown, when they reach the desired maturity, and, optionally, in separating the seeds from the harvested plants.

According to the invention, the term “agrochemical composition” is intended to mean any agrochemical composition comprising at least one active product having one of the following activities, herbicidal, fungicidal, bactericidal, virucidal or insecticidal.

According to a preferential embodiment of the growing method according to the invention, the agrochemical composition comprises at least one active product having at least one fungicidal and/or bactericidal activity, more preferentially exhibiting an activity complementary to that of the termicin produced by the transformed plants according to the invention.

According to the invention, the expression “product exhibiting an activity complementary to that of the termicin” is intended to mean a product exhibiting a complementary spectrum of activity, i.e. a product which will be active against attacks from contaminants (fungi, bacteria or viruses) insensitive to the termicin, or else a product the spectrum of activity of which totally or partly covers that of the termicin, and the dose of application of which will be substantially decreased due to the presence of the termicin produced by the transformed plant.

The termicin is a peptide which is particularly active against fungi and yeasts and certain bacteria, and may, in this respect, be used in a preventative or curative capacity to protect various organisms against fungal and/or bacterial attacks. The present invention therefore also relates to the termicin as a medicinal product. It also relates to the use of the termicin for treating the plants against fungal and/or bacterial attacks, by applying the termicin directly to said plants.

The present invention also relates to a composition comprising a termicin according to the invention and an appropriate vehicle. The primary quality of the appropriate vehicle is that it does not substantially degrade the termicin in the composition, and it does not decrease the bactericidal and fungicidal properties of the termicin. The term “vehicle” is intended to mean any substance which is added to the termicin in the present composition in order to promote essentially the transport and the protection of said termicin. This composition may be a cosmetic composition and, in this case, the appropriate vehicle is cosmetically acceptable, also suitable for application to the skin or superficial skin growths. The composition may also be a pharmaceutical composition for therapeutic use in human or animal health, and in this case, the appropriate vehicle is pharmaceutically acceptable, appropriate for administration of the termicin topically, per os, or by injection. According to another embodiment of the invention, the composition may be an agrochemical composition and, in this case, the appropriate vehicle is agrochemically acceptable, appropriate for application to the plants or in the proximity of the plants, without degrading them. According to another embodiment of the invention, the composition may be a food composition for animal or human nutrition, and in this case, the appropriate vehicle is nutritionally acceptable, i.e. compatible with assimilation of the composition by ingestion.

The examples below make it possible to illustrate the present invention without, however, limiting the scope thereof.

### EXAMPLE I

### Isolation and Characterization of th Termicin From Whole bodies or From Blood Cells of Naive or Immunized Imagos (Male or Female) of th isopt ra

Pseudacanthotermes spiniger

### EXAMPLE.I1

### Isolation of the Termicin 1-1 Induction of the Biological Synthesis of an Antifungal Substance in the Hemolymph of

Pseudacanthotermes spiniger

The imagos of the fungus-forming isoptera Pseudacanthotermes spiniger were immunized with an injection of 2 μl of a mixture of 2500 cells of Micrococcus luteus (Gram-positive) and 2500 cells of Escherichia coli 1106 (Gram-negative) prepared from cultures produced in Luria-Bertani medium for 12 hours at 37° C. The animals thus infected were in a humid chamber containing compost and maintained at 25° C., for 24 h. The animals are sacrificed by freezing and conserved in the freezer until use.

1-2 Preparation of the Extract

The termites (whole bodies) are reduced to a fine powder in a mortar in the constant presence of liquid nitrogen. An extract can be prepared both on male and female animals, naive or immunized. An extract from blood cells or from salivary glands is also possible.

1-3 Acidification of the Extract

After slow reheating of the powder obtained, a 0.1% trifluoroacetic acid solution is added slowly in order to obtain a pH close to 3 for the extract. The solution which makes it possible to produce the extract contains, besides the trifluoroacetic acid, a protease inhibitor (aprotinin at a final concentration of 10 μg/ml) and a melanization inhibitor (phenylthiourea at 20 μM). The extraction of the peptide under acid conditions was carried out for 30 min with slight agitation in a bath of iced water. The extract obtained was then centrifuged at 6° C. for 30 min at 14 000 g.

I-4 Purification of the Peptides

a) Prepurification By Solid-phas Extraction

An amount of extract equivalent to 200 individuals was loaded onto a reverse-phase support, as marketed in the form of a cartridge (Sep-Pak™ C₁₈, Waters Associates), equilibrated with acidified (0.05% TFA) water. The hydrophilic molecules were removed by simple washing with acidified water. The peptide was eluted with a solution of acetonitrile at 40% prepared in 0.05% TFA. The fraction eluted at 40% of acetonitrile was dried under vacuum with the aim of removing the acetonitrile and the TFA, and was then reconstituted in sterile ultrapure water before being subjected to the first purification step. It is also possible to perform the elution of the peptide with concentrations of acetonitrile in acidified medium of between 20% and 100%.

b) Purification By High Performance Liquid Chromatography (HPLC) On a Reverse-phase Column and a Size Exclusion Column

First step: the fraction containing the peptide was analyzed by reverse-phase chromatography on a semipreparative Aquapore RP-300 C₈ column (Brownlee™, 220×7 mm, 300 Å), the elution was performed with a linear gradient of acetonitrile of 2 to 60% in 0.05% TFA for 120 minutes at a constant flow rate of 1.3 ml/min. The fractions were collected manually by following the variation in absorbance at 214 nm and 225 nm. The fractions collected were dried under vacuum, reconstituted with ultrapure water and analyzed for their antimicrobial activity using the assays described below.

Second step: the antimicrobial fraction corresponding to the peptide was analyzed on two size exclusion columns mounted in series (Ultraspherogel SEC 3000 and SEC 2000, 7.5×300 mm, Beckman™) and protected by a precolumn (Ultraspherogel SEC, 7.5×40 mm, Beckman™).

The elution is performed under isocratic conditions with 30% of acetonitrile in the presence of 0.05% TFA at a flow rate of 0.4 ml/min. The fractions are harvested as a function of the variation in optical density measured at 225 and 214 nm. The fractions collected were dried under vacuum, reconstituted with ultrapure water and analyzed for their antimicrobial activity using the assays described below.

Third step: the antimicrobial fraction corresponding to the peptide was analyzed on the same reverse-phase column as for the initial step, using as elution conditions a discontinuous linear gradient of acetonitrile in acidified (0.05% TFA) water of 2-15% in 10 min and of 15-45% in 120 min. The fractions are collected and analyzed for their antimicrobial activity as previously.

Fourth step: the antimicrobial fraction is analyzed on an Aquapore OD-300 (220×4.6 mm, Brownlee™) reverse-phase analytical column, and the elution of the compound of interest is performed with a biphasic linear gradient of acetonitrile in acidified water of 2-15% in 10 min followed by a gradient 22-32% in 50 min at a flow rate of 0.8 ml/min and at a controlled temperature of 30° C.

Final purification step: the final purification step is carried out on a reverse-phase column with a small internal diameter, termed “narrow bore” column (Delta Pak HPIC₁₈, 2×150 mm, Waters™) at a temperature controlled at 30° C. at a flow rate of 0.2 ml/min. The gradient used to perform this final purification step is a biphasic linear gradient of acetonitrile in acid medium (0.05% TFA) of 2-17% in 10 min and of 17-27% in 40 min. The conditions for fractionation and also for determination of the antimicrobial activity are those described for the prior steps (steps 1 to 4, above).

### EXAMPLE I.2

### Structural Characterization of the Termicin

2-1 Verification of the Purity By Capillary Zon Electrophoresis

The purity of the antifungal peptide was verified by capillary zone electrophoresis on a 270-HT model (PEApplied Biosystems division of Perkin Elmer). 1 nl of a solution containing 50 μM of purified peptide was introduced into a silica capillary (72 cm×50 μm) by vacuum-assisted injection and the analysis was carried out in 20 mM citrate buffer at pH 2.5. The electrophoresis was carried out at 20 kV from-the anode to the cathode for 20 minutes at 30° C. The migration was recorded at 200 nm.

2-2 Determination of the Number of Cysteines: Reduction and S-pyridylethylation

The number of cysteine residues was determined on the native peptide by reduction and S-pyridylethylation. 1 nanomole of native peptide was reduced in 40 μl of 0.5 M Tris/HCl buffer, pH 7.5, containing 2 mM of EDTA and 6 M of guanidinium chloride, in the presence of 2 μl of 2.2 M dithiothreitol. The reaction medium was placed under a nitrogen atmosphere. After incubation for 60 min in the dark, 2 μl of freshly distilled 4-vinylpyridine were added to the reaction, which was then incubated for 10 min at 45° C. in the dark under a nitrogen atmosphere. The pyridylethylated peptide was then separated from the constituents of the reaction medium by reverse-phase chromatography using a linear gradient of acetonitrile in the presence of 0.05% TFA.

2-3 Determination of the Mass of the Native Peptide, of the S-pyridylethylated Peptide and of the Proteolytic Fragments By MALDI-TOF (Matrix Assisted Laser Desorption Ionization-Tim Of Flight) mass sp ctrometry

The mass measurements were performed on a Bruker Biflex™ III MALDI-TOF mass spectrometer (Bremen, Germany) in the linear positive mode. The mass spectra were calibrated externally with a standard mixture of peptides of known m/z, respectively 2199.5 Da, 3046.4 Da and 4890.5 Da. The various products to be analyzed were deposited onto a fine layer of α-cyano-4-hydroxycinnamic acid crystals, obtained by rapid evaporation of a saturated solution in acetone. After drying under a slight vacuum, the samples were washed with a drop of 0.1% trifluoroacetic acid before being introduced into the mass spectrometer.

2-4 Sequencing By Edman Degradation

Automatic sequencing by Edman degradation of the native peptide, of the S-pyridylethylated peptide and of the various fragments obtained after the various proteolytic cleavages, and detection of the phenylthiohydantoin derivatives were carried out on an ABI473A sequencer (PEApplied Biosystems division of Perkin Elmer).

2-5 Proteolytic Cleavages

Confirmation of the peptide sequence in the C-terminal region

600 pmol of reduced and S-pyridylethylated peptide were incubated in the presence of endoproteinase-Arg-C at a ratio of 1:50 by weight:weight (specific cleavage of arginine residues on the C-terminal side, Takara, Otsu) according to the conditions recommended by the supplier (10 mM Tris HCl, pH 8, in the presence of 0.01% Tween 20). The incubation is carried out for 16 h at 37° C. After the reaction has been stopped with 0.05% TFA, or any other acid solution with a final concentration of less than 3%, the peptide fragments were separated by reverse-phase HPLC on a column of the Narrowbore Delta-Pak™ HPIC₁₈ type (Waters Associates, 150×2 mm) in a linear gradient of acetonitrile of 0 to 60% in 90 min in 0.05% TFA with a flow rate of 0.2 ml/min and a constant temperature of 30° C. The fragments obtained were analyzed by MALDI-TOF mass spectrometry and the peptide corresponding to the C-terminal fragment was sequenced by Edman degradation.

Determination of the disulfide bridge arrangement by proteolysis with thermolysin

The native peptide (6 μg) was incubated for 1 hour in the presence of 3 μg of thermolysin (Boehringer Mannheim, 1/2 thermolysin/peptide ratio, weight:weight) at 37° C. in 0.1 M MES (N-ethylmorpholine) buffer at pH 7 in the presence of 2 mM of CaCl₂. The reaction was stopped by adding formic acid and the reaction products were immediately separated by reverse-phase chromatography on a Narrowbore Delta-Pak™ HPIC₁₈ column (Waters Associates, 150×2.2 mm) in a linear gradient of acetonitrile of 2 to 50% in 100 min in 0.05% TFA, at a flow rate of 0.2 ml/min at 30° C., preceded by an isocratic step with 2% of acetonitrile for 10 min. The fragments obtained were analyzed by MALDI-TOF mass spectrometry and sequenced by Edman degradation.

### EXAMPLE II

### Expression of Termicin in the Yeast Saccharomyces cerevisiae

All the techniques used hereafter are standard laboratory techniques. The detailed protocols of these techniques have been described in particular in Ausubel et al. (1987, Current Protocols in Molecular Biology, eds. Greene, Publ. Wiley & Sons).

### EXAMPLE II-1

### Assembly of the Synthetic Gene

The assembly was carried out using 6 synthetic oligonucleotides encoding the 36 amino acids of the termicin, preceded by the 5 C-terminal amino acids of the pre-pro sequence of the yeast factor al (Mfa1) and followed by the additional peptide residue consisting of the glycine amino acid (SEQ ID No. 3). The oligonucleotides were chosen by taking into account the preferential codons used by S. cerevisiae.

The assembly took place in several steps:

oligonucleotides 2 to 5 were phosphorylated at their 5′ ends via the action of polynucleotide kinase (New England Biolabs);

oligonucleotides 1 to 6 were mixed, heated to 100° C. and hybridized by slowly decreasing the temperature to 25° C. over 3 hours;

the hybridized oligonucleotides were subjected to treatment with T4 bacteriophage ligase (New England Biolabs) for 15 hours at 15° C.;

the block of DNA resulting from the hybridization of the oligonucleotides represented in FIG. 1, bordered by the HinDIII and Sal I restriction sites, was inserted into the plasmid pBluescript SK+ (Stratagene) digested with the HinDIII and Sal I enzymes. The ligation mixture was then used to transform competent E. coli DH5α cells (Stratagene). Several clones were analyzed and sequenced. One of these clones which had the desired sequence was called pJL187.

### EXAMPLE II-2

### Construction of the Vector Which Allows Secretion of the Synthesized Termicin

The HinDIII-SalI DNA fragment of the vector pJL187, carrying the sequence encoding the termicin, and also the SphI-HinDIII fragment of the vector M13JM132 (Michaut et al., 1985. FEBS Letters, 395, pp. 6-10, Lamberty et al., 1999, JBC, 274, pp. 9320-9326) were inserted between the SphI and SalI sites of the plasmid pTG4812 (Michaut et al., 1996, FEBS Letters., 395, pp. 6-10, Lamberty et al., 1999, JBC, 274, pp. 9320-9326). The SphI-HinDIII fragment of the vector M13JM132 contains the sequence of the promoter of the yeast MFα1 gene and also the sequence encoding the pre-pro region of the MFα1 factor. In the resulting plasmid, pJL193, the synthetic termicin gene is therefore found to be inserted between the pre-pro sequences of the Mfα1 factor and the transcription terminator; this construct should therefore ensure maturation and secretion of the termicin.

### EXAMPLE II-3

### Transformation of an S. cerevisiae Strain With the DNA of the Plasmid pSEA2 and Analysis of the Transformants

The yeast strain TGY 48.1 (MATa, ura3-D5 his, pra1, prb1, prc1, cps1; Reichhart et al., 1992, Invert. Reprod. Dev. 21, pp. 15-24) was transformed with the plasmid pJL193. The transformants were selected at 29° C. on a selective YNBG medium (0.67% yeast nitrogen base, 2% glucose), supplemented with 0.5% of casamino acids and not containing any uracil. After transformation, several yeast clones, selected for the ura+ characteristic, were cultured for 48 h at 29° C. in 50 ml of selective medium.

After centrifugation (4000 g, 30 min, 4° C.), the supernatant was acidified to pH 3.5 with acetic acid, before being loaded onto a Sep-Pak™ C₁₈ cartridge (Waters Associates) equilibrated with acidified (0.05% TFA) water. The various proteins attached to the cartridge were eluted with solutions of 0.05% TFA containing increasing percentages of acetonitrile.

The 45% fraction, exhibiting antimicrobial activity, was concentrated under vacuum and subjected to a step of purification by HPLC analysis on an Aquapore RP-300 C₈ reverse-phase analytical column (Brownlee™, 220×4.6 mm, 300 Å), using a linear gradient of acetonitrile of 2% to 40% in 80 min and 17 to 27% in 60 min in 0.05% TFA with a constant flow rate of 0.8 ml/min. The fractions were collected manually by following the variation in absorbance at 225 nm and 254 nm. The fractions collected were dried under vacuum, reconstituted with ultrapure water and analyzed for their antimicrobial activity under the conditions described in Example III. The structural characterization of the peptide was carried out as described in Example I.2.

### EXAMPLE II-4

### Production of Recombinant Termicin on a Semipreparativ Scale

One of the transformed yeast clones expressing the termicin was cultured at 29° C. for 24 h in 100 ml of selective medium. This preculture was then used to inoculate 4 l of selective medium and the culture was prepared for 48 h at 29° C. The yeasts were removed by centrifugation (4000 g, 30 min, 4° C.). After centrifugation (4000 g, 30 min, 4° C.), the supernatant was acidified to pH 3.5 with acetic acid, before being loaded onto a Sep-Pak™ C₁₈ cartridge (Waters Associates) equilibrated with acidified (0.05% TFA) water. The various proteins attached to the cartridge were eluted with solutions of 0.05% TFA containing increasing percentages of acetonitrile.

The 45% fraction, exhibiting antimicrobial activity, was concentrated under vacuum and subjected to a step of purification by solid-phase extraction on a cation exchange cartridge of the Sep-Pak CM type, equilibrated in 25 mM of ammonium acetate buffer at pH 3.6. The recombinant peptide was eluted with a solution at high ionic strength (for example 1M of sodium chloride) in 25 mM ammonium carbonate buffer at pH 3.6. The recombinant peptide was desalified on a Sep-Pak™ C₁₈ cartridge (Waters Associates) equilibrated with acidified (0.05% TFA) water. The recombinant peptide retained on the cartridge was then eluted with a solution of acetonitrile in acidified (0.05% TFA) water; a solution comprising 45% acetonitrile is advantageous. The fraction containing the peptide was purified by HPLC analysis on an Aquapore RP-300 C₈ reverse-phase preparative column (Brownlee™, 250×10 mm, 300 Å), using a discontinuous linear gradient of acetonitrile of 2% to 17% in 10 min and of 17% to 27% in 60 min, in 0.05% TFA with a constant flow rate of 2.5 ml/min. The fractions were collected manually by following the variation in absorbance at 225 nm and 254 nm. The fractions collected were dried under vacuum, reconstituted with ultrapure water and analyzed for their antimicrobial activity under the conditions described in Example III. The structural characterization of the peptide was carried out as described in Example I.2.

### EXAMPLE III

### In Vitro Activity Assay: Measurement of Antimicrobial Activity By Microspectrophotometry

This methodology was used to demonstrate the antimicrobial molecules in the course of the various purification steps, to determine the spectrum of activity of the peptide and to determine the minimum inhibitory concentration (MIC) at which the peptide was active. The MIC was expressed as a concentration range [a]-[b], in which [a] was the minimal concentration at which the start of growth was observed and [b] the concentration for which no growth was observed. Examples of the specific activity of the termicin, with respect to filamentous fungi, to yeasts and to bacteria, are given in Tables 1 to 4.

### EXAMPLE III-1

### Assay for Detecting Activity Against Filamentous Fungi

The antifungal activity was detected using a growth inhibition assay in liquid medium. The spores of the fungi to be tested were suspended in a culture medium of the “potato-glucose” type. Preferably, 12 g of Potato Dextrose Broth medium (Difco) per liter of demineralized water are used. Two antibiotics were added to the culture medium: tetracycline (final concentration of 10 μg/ml) and cefotaxim (100 μg/ml). 10 μl of each fraction to be analyzed were deposited into microtitration plates in the presence of 90 μl of culture medium containing the spores (at a final concentration of 104 spores/ml). The incubation was carried out in a humid chamber at 30° C. for 48 hours. The fungal growth was observed under a photon microscope after 24 h and quantified after 48 h by measuring the absorbance at 600 nm using a microtitration plate reader spectrophotometer.

Filamentous fungi tested: Tricophyton mentagrophytes (gift from Dr H. Koenig, Hôpital civil, Strasbourg); Nectria haematococca, Fusarium culmorum, Trichoderma viride (mycothéque [fungi collection] of the Université Catholique de Leuven [Catholic University of Leuven], Belgium); Neurospora crassa, Fusarium oxysporum (mycothéque [fungi collection] of the company Clause, Paris).

The results of the assay for termicin activity against filamentous fungi are given in Table 1 below.

TABLE 1
Termicin activity against filamentous fungi

| Fungi | MIC of the termicin (μM) |
|---|---|
| Neurospora crassa | 0.2-0.4 |
| Fusarium culmorum | 0.2-0.4 |
| Fusarium oxysporum | 0.8-1.5 |
| Nectria haematococca | 0.05-0.1 |
| Trichoderma viride | 6-12 |
| Tricophyton mentagrophytes | 6-12 |

TABLE 2
Termicin activity against phytopathogenic
fungi. The activity is expressed here by a percentage
growth corresponding to 100 × (1 = absorbance with
product/absorbance of the control without product), the
absorbance being measured at 600 rm, 5 days after the
beginning of the experiment

| | Termicin 1 + | | | | | |
|---|---|---|---|---|---|---|
| | 1 glycine | | | Termicin 2 | | |
| | 40 ppm | 20 ppm | 10 ppm | 40 ppm | 20 ppm | 10 ppm |
| Cercospora | 95% | 68% | 65% | 90% | 70% | 70% |
| fijensis | | | | | | |
| Botrytis | 30% | 10% | 4% | 60% | 30% | 10% |
| cinerea | | | | | | |
| Septoria | 30% | 20% | 4% | 40% | 30% | 20% |
| nodorum | | | | | | |
| Septoria | 85% | 80% | 60% | 90% | 70% | 70% |
| tritici | | | | | | |
| Rhizoctonia | 34% | 0% | 0% | 60% | 60% | 40% |
| solani | | | | | | |
| Fusarium | 90% | 65% | 20% | 95% | 60% | 45% |
| germinearum | | | | | | |
| Fusarium | 40% | 30% | 30% | 60% | 50% | 45% |
| nivale | | | | | | |

The results in Table 2 show considerable activity on the phytopathogenic fungi.

### EXAMPLE III-2

### Assay For Detecting Activity Against Yeasts

The various yeast strains were incubated in a culture medium of the “Sabouraud” type, and incubated at 30° C. for 24 h with slow shaking. The sample to be tested (10 ∞l) was deposited into wells of a microtitration plate, to which were added 90 μl of a dilute yeast culture, the density of which was adjusted to OD 600=0.001. The growth was evaluated by measuring the absorbance at 600 nm using a microtitration plate reader spectrophotometer.

Yeasts tested: Candida albicans, Cryptococcus neoformans, Saccharomyces cerevisiae (gift from Dr H. Koenig, Hôpital civil, Strasbourg).

The results of the assay for termicin activity against yeasts are given in Table 3 below.

TABLE 3
Termicin activity against yeasts

| Yeasts | MIC of the termicin (μM) |
|---|---|
| Candida albicans | 6-12 |
| Cryptococcus neoformans | 6-12 |
| Saccharomyces cerevisiae | 6-12 |

The results of Examples III-1 and III-2 show the excellent antifungal activity of the peptide according to the invention.

### EXAMPLE III-3

### Assay For Detecting Activity Against Bacteria

The antibacterial activity was detected using a growth inhibition assay in liquid medium. The bacteria to be tested were suspended in a nutritive medium of the “Poor Broth” or “Luria Bertani” type. Preferably, use is made of a solution of 10 g/l of bactotryptone supplemented with 5 g/l of NaCl prepared in demineralized water for the “Poor Broth” and a solution of 10 g/l of bactotryptone, 10 g/l of NaCl, and 5 g/l of yeast extract, at pH 7.4, for the Luria Bertani medium. 10 μl of each fraction to be analyzed are deposited into microtitration plates in the presence of 90 μl of culture medium containing the bacteria (at a final concentration equivalent to 1 m OD at 600 nm). The incubation was carried out at 25° C. for 12 to 24 hours. The bacterial growth was measured by following absorbance at 600 nm using a microtitration plate reader spectrophotometer.

Bacteria tested: Bacillus megaterium and Micrococcus luteus (collection of the Pasteur Institute of Paris); Aerococcus viridans, Staphylococcus aureus and Streptococcus pyrogenes (gift from Prof. Monteil, Institute of Bacteriology, Université Louis Pasteur of Strasbourg) for the Gram-positive strains, and Escherichia coli D22, E. coli SBS363 (gift from Mr Boquet of the Centre d'Etudes Nucléaires [Center for Nuclear Studies] of Saclais) and Pseudomonas aeruginosa for the Gram-negative microorganisms.

The results of the assay for termicin activity against bacteria, when they were found to be sensitive, are given in Table 4 below.

TABLE 4
Termicin activity against Gram-positive
bacteria

| Bacteria | MIC of the termicin (μM) |
|---|---|
| Streptococcus pyrogenes | 25-50 |
| Micrococcus luteus | 50-100 |
| Bacillus megaterium | 50-100 |

The results of Example III-3 show activity against Gram-positive bacteria.

1. An isolated polynucleotide encoding a termicin.

2. The polynucleotide as claimed in claim 1, characterized in that it comprises a polynucleotide selected from the group consisting of:
(a) an isolated polynucleotide encoding the peptide described by the sequence identifier SEQ ID No. 2
(b) an isolated polynucleotide which hybridizes to the polynucleotide according to (a)
(c) an isolated polynucleotide homologous to a polynucleotide according to (a) or (b)
(d) a fragment of a polynucleotide according to (a), (b) or (c).

3. The polynucleotide as claimed in claim 2, characterized in that it comprises a polynucleotide represented by the sequence identifier SEQ ID No. 1.

4. An isolated polynucleotide comprising a polynucleotide as claimed in one of claims 1 to 3.

5. An antimicrobial peptide of the family of defensins, characterized in that it is encoded by a polynucleotide as claimed in one of claims 1 to 4.

6. The antimicrobial peptide as claimed in claim 5, characterized in that it comprises the amino acid sequence described by the sequence identifier SEQ ID No. 2.

7. A chimeric gene comprising, functionally linked to one another, at least:
(a) one promoter which is functional in a host organism
(b) a polynucleotide as claimed in one of claims 1 to 4
(c) a terminator element which is functional in a host organism.

8. The chimeric gene as claimed in claim 7, characterized in that the promoter is a constitutive promoter.

9. The chimeric gene as claimed in claim 7, characterized in that the promoter is an inducible promoter.

10. The chimeric gene as claimed in one of claims 7 to 9, characterized in that it also comprises a signal peptide or a transit peptide which is functional in said host organism.

11. The chimeric gene as claimed in one of claims 7 to 10, characterized in that the host organism is a microorganism.

12. The chimeric gene as claimed in claim 11, characterized in that the microorganism is a yeast.

13. The chimeric gene as claimed in one of claims 7 to 10, characterized in that the host organism is a plant cell or a plant.

14. An expression or transformation vector containing a chimeric gene as claimed in one of claims 7 to 13.

15. The vector as claimed in claim 14, characterized in that it is a plasmid, a phage or a virus.

16. A host organism transformed with one of the vectors as claimed in either of claims 14 and 15.

17. The host organism as claimed in claim 16, characterized in that it is a microorganism.

18. The host organism as claimed in claim 17, characterized in that the microorganism is a bacterium of the species Escherichia coli.

19. The host organism as claimed in claim 17, characterized in that the microorganism is a yeast of the Saccharomyces, Kluyveromyces or Pichia genus.

20. The host organism as claimed in claim 17, characterized in that the microorganism is a baculovirus.

21. A transformed plant cell containing a chimeric gene as claimed in one of claims 7 to 13.

22. A transformed plant containing a chimeric gene as claimed in one of claims 7 to 13.

23. The transformed plant as claimed in claim 22, characterized in that it is resistant to fungal diseases such as those caused by fungi of the Cercospora genus, in particular Cercospora fijensis, of the Septoria genus, in particular Septoria nodorum or Septoria tritici, of the Fusarium genus, in particular Fusarium nivale or Fusarium graminearum, of the Botrytis genus, in particular Botrytis cinerea, or of the Rhizoctonia genus, in particular Rhizoctonia solani.

24. A part of a plant as claimed in claim 23.

25. A seed of a plant as claimed in claim 23.

26. A method for producing a peptide as claimed in either of claims 5 and 6, characterized in that it comprises the steps of:
(a) culturing a transformed host organism as claimed in one of claims 16 to 20 or a plant cell as claimed in claim 21, in a suitable culture medium,
(b) extracting and purifying, totally or partially, the peptide as claimed in either of claims 5 and 6, produced during step (a) by the transformed host organism.

27. A composition comprising an antimicrobial peptide as claimed in either of claims 5 and 6, and an appropriate vehicle.

28. A method for protecting plants against phytopathogenic organisms, characterized in that a termicin as claimed in either of claims 5 and 6 is expressed in said plants by transforming said with a vector as claimed in either of claims 14 and 15.

29. A method for growing transformed plants as claimed in either of claims 22 and 23, characterized in that it consists in planting the seeds of said transformed plants in an area of a field suitable for growing said plants, in applying to said area of said field an agrochemical composition, without substantially affecting said seeds or said transformed plants, then harvesting the plants grown, when they reach the desired maturity, and, optionally, in separating the seeds from the harvested plants.

30. The growing method as claimed in claim 29, characterized in that the agrochemical composition comprises at least one active product having at least one fungicidal and/or bactericidal activity.

31. The growing method as claimed in claim 30, characterized in that the active product exhibits an activity complementary to that of the peptide as claimed in either of claims 5 and 6.


SEQUENCE LISTING (4 sequences)

SEQ ID NO: 1 - DNA, 112 bp, Pseudacanthotermes spiniger
  CDS (1)..(108)
gcttgtaatttccaatcttgttgggccacgtgtcaagctcaacattctatttactttagaagagctttctgtgatagatctcaatgtaaatgtgtttttgttagaggttaag

SEQ ID NO: 2 - protein, 36 aa, Pseudacanthotermes spiniger
ACNFQSCWATCQAQHSIYFRRAFCDRSQCKCVFVRG

SEQ ID NO: 3 - DNA, 127 bp, Artificial
  Mfalpha1 Signal peptide and termicin
agcttggataaaagagcttgtaatttccaatcttgttgggccacgtgtcaagctcaacattctatttactttagaagagctttctgtgatagatctcaatgtaaatgtgtttttgttagaggttaag

SEQ ID NO: 4 - protein, 41 aa, Artificial
  Mfalpha1 Signal peptide and termicin
SLDKRACNFQSCWATCQAQHSIYFRRAFCDRSQCKCVFVRG